(19) United States
(12) Patent Application Publication
(10) Pub. No.: US 2022/0120751 A1
(43) Pub. Date: Apr. 21, 2022

(54) METHOD FOR DIAGNOSING OESOPHAGOGASTRIC CANCER

(71) Applicant: IP2IPO Innovations Limited, London (GB)

(72) Inventors: George Hanna, London (GB); Piers Boschier, London (GB)

(21) Appl. No.: 17/430,639

(22) PCT Filed: Feb. 12, 2020
(86) PCT No.: PCT/GB2020/050317

§ 371 Date: Aug. 12, 2021

(30) Foreign Application Priority Data
Feb. 13, 2019 (GB) 1901991.8

(51) Int. Cl. G01N 33/574; G01N 33/497

(52) U.S. Cl. CPC G01N 33/57446; G01N 2033/4975; G01N 33/57484; G01N 33/497

(57) ABSTRACT

The invention relates to a method for diagnosing or for providing a prognosis of a subject suffering from oesophagogastric cancer, or a pre-disposition thereto. The method comprises analysing, in an endoluminal sample obtained from a test subject, the level of at least one biomarker compound selected from the group consisting of: acetone, acetic acid, butyric acid, pentanoic acid and hexanoic acid. The method further comprises comparing this level with a reference for the level of the at least one biomarker compound in an individual who does not suffer from oesophagogasatric cancer. In particular, an increase in the concentration of the at least one biomarker compound, in the endoluminal sample from the test subject, compared to the reference, suggests that the subject is suffering from oesophagogastric cancer, or has a pre-disposition thereto, or provides a negative prognosis of the subject's condition.

The present invention relates to cancer, and particularly although not exclusively, to detecting volatile organic compounds (VOCs) for diagnosis of, and prognostication in, oesophagogastric cancer. In addition, the invention relates to a novel apparatus for detecting VOCs for oesophagogastric cancer, and diagnostic and prognostic methods of using such apparatus.

The chemical analysis of volatile organic compounds (VOCs) in humans is a rapidly evolving field that has the potential to contribute to the non-invasive detection of multiple disease states. A recent systematic review on the diagnostic accuracy of VOC-based exhaled breath tests showed their potential for non-invasive cancer detection.¹ Previous studies have reported higher concentrations of specific VOCs, within the exhaled breath, gastric content and urine of patients with oesophagogastric cancer.¹⁻⁵ However, whilst several studies have suggested a role for these VOCs in important regulatory processes in oesophagogastric cancer,^{6,7} many of the biochemical pathways relating to their origin in humans are as yet unknown. Nevertheless, it has been postulated that the deregulated production of specific VOCs occurs directly from cancer tissues, and these VOCs may pass in to the systemic circulation with subsequent partition across the alveolar-capillary barrier. Alternatively, VOCs may be released directly by the mucosa within the aerodigestive tract.^{8,9} National studies have shown that about 9% of gastric and oesophageal cancers were missed during endoscopy prior to diagnosis (30-31). Accordingly, there is a need for improved techniques for diagnosing oesophagogastric cancer.

The present invention arises from the inventor's work in trying to overcome the problems associated with the prior art.

The inventors have investigated the production of targeted VOCs in oesophagogastric cancer through analysis within different anatomical compartments, including mixed breath, isolated bronchial breath and oesophagogastric luminal air, and have observed significant differences in the relative abundance of VOCs within these three compartments. Indeed, the inventors were surprised to observe that the concentration of the VOC biomarkers for oesophagogastric cancer in oesophagogastric luminal air (also known as endoluminal gastric gas) is significantly higher than both the mixed breath and the bronchial breath, and that there is little or no difference between the bronchial and mixed breath. Accordingly, the inventors now have a much clearer understanding of the source of origin of the VOCs, which were detected, and their association with oesophagogastric cancer for use as diagnostic or prognostic biomarkers. The clinical implication of these results is to use endoluminal gas sampling to provide a gas biopsy for the detection of cancer, and in particular oesophagogastric cancer.

Hence, in a first aspect, there is provided a method for analysing an endoluminal sample from a test subject, the method comprising:
(i) determining, in an endoluminal sample from the test subject, the level of at least one biomarker compound selected from the group consisting of: acetone, acetic acid, butyric acid, pentanoic acid and hexanoic acid; and
(ii) comparing the level of the at least one biomarker compound determined in step (i) to at least one control level, wherein the levels of the at least one compound are indicative of whether the subject has oesophagogastric cancer.

In a second aspect, there is provided a method for diagnosing a subject suffering from oesophagogastric cancer, or a pre-disposition thereto, or for providing a prognosis of the subject's condition, the method comprising analysing, in an endoluminal sample obtained from a test subject, the level of at least one biomarker compound selected from the group consisting of: acetone, acetic acid, butyric acid, pentanoic acid and hexanoic acid, and comparing this level with a reference for the level of the at least one biomarker compound in an individual who does not suffer from oesophagogastric cancer, wherein an increase in the concentration of the at least one biomarker compound, in the endoluminal sample from the test subject, compared to the reference, suggests that the subject is suffering from oesophagogastric cancer, or has a pre-disposition thereto, or provides a negative prognosis of the subject's condition.

In a third aspect, there is provided an apparatus for diagnosing a subject suffering from oesophagogastric cancer, or a pre-disposition thereto, or for providing a prognosis of the subject's condition, the apparatus comprising:—
means for analysing, in an endoluminal sample obtained from a test subject, the level of at least one biomarker compound selected from the group consisting of: acetone, acetic acid, butyric acid, pentanoic acid and hexanoic acid; and
a reference for the concentration of the at least one biomarker compound in a sample from an individual who does not suffer from oesophagogastric cancer,
   wherein the apparatus is used to identify an increase in the concentration of the at least one biomarker compound in the endoluminal sample from the test subject, compared to the reference, thereby suggesting that the subject suffers from oesophagogastric cancer, or has a pre-disposition thereto, or provides a negative prognosis of the subject's condition.

Methods of the first and second aspect may comprise administering or having administered, to the subject, a therapeutic agent or putting the subject on a specialised diet, wherein the therapeutic agent or the specialised diet prevents, reduces or delays progression of oesophagogastric cancer.

Hence, according to a fourth aspect of the invention, there is provided a method of treating an individual suffering from oesophagogastric cancer, said method comprising the steps of:
(i) determining, in an endoluminal sample obtained from a test subject, the level of at least one biomarker compound selected from the group consisting of: acetone, acetic acid, butyric acid, pentanoic acid and hexanoic acid, wherein an increase in the level of the at least one biomarker compound in the endoluminal sample from the test subject compared to the reference suggests that the subject is suffering from oesophagogastric cancer, or has a pre-disposition thereto, or has a negative prognosis; and
(ii) administering, or having administered, to the test subject, a therapeutic agent or putting the test subject on a specialised diet, wherein the therapeutic agent or the specialised diet prevents, reduces or delays progression of oesophagogastric cancer.

In a fifth aspect, there is provided a method for determining the efficacy of treating a subject suffering from oesophagogastric cancer with a therapeutic agent or a specialised diet, the method comprising analysing, in an endoluminal sample obtained from a test subject, the level of at least one biomarker compound selected from the group consisting of: acetone, acetic acid, butyric acid, pentanoic acid and hexanoic acid, and comparing this level with a reference for the level of at least one biomarker compound in an individual who does not suffer from oesophagogastric cancer, wherein:
(i) a decrease in the level of the at least one biomarker compound in the endoluminal sample from the test subject, compared to the reference, suggests that the treatment regime with the therapeutic agent or the specialised diet is effective, or
(ii) an increase in the concentration of at least one biomarker compound in the endoluminal sample from the test subject, compared to the reference, suggests that the treatment regime with the therapeutic agent or the specialised diet is ineffective.

In a sixth aspect, the invention provides an apparatus for determining the efficacy of treating a subject suffering from oesophagogastric cancer with a therapeutic agent or a specialised diet, the apparatus comprising:—
means for analysing, in an endoluminal sample obtained from a test subject, the level of at least one biomarker compound selected from the group consisting of: acetone, acetic acid, butyric acid, pentanoic acid and hexanoic acid; and
a reference for the concentration of the at least one biomarker compound in a sample from an individual who does not suffer from oesophagogastric cancer,
wherein the apparatus is used to identify:
(i) a decrease in the level of the at least one biomarker compound in the endoluminal sample from the test subject, compared to the reference, thereby suggesting that the treatment regime with the therapeutic agent or the specialised diet is effective; or
(ii) an increase in the concentration of the at least one biomarker compound in the endoluminal sample from the test subject, compared to the reference, thereby suggesting that the treatment regime with the therapeutic agent or the specialised diet is ineffective.

In a seventh aspect, there is provided use of at least one biomarker compound selected from the group consisting of: acetone, acetic acid, butyric acid, pentanoic acid and hexanoic acid in or from an endoluminal sample obtained from a test subject, as a biomarker for diagnosing a subject suffering from oesophagogastric cancer, or a pre-disposition thereto, or for providing a prognosis of the subject's condition.

Advantageously, the methods and apparatuses of the invention enable targeted and accurate quantification of the at least one biomarker compound within the headspace of oesophagogastric tissue. As shown in Table 2, by detecting at least biomarker compound in an endoluminal sample (instead of in mixed breath and/or isolated bronchial breath), the inventors believe that it will be possible to diagnose many more cases of oesophagogastric cancer. The inventors believe that the biomarkers, which are volatile fatty acids, reach the breath through systemic circulation, and not up through the lumen of the gastrointestinal track to the mouth. Obtaining gas from the gastrointestinal lumen for cancer detection, therefore, has never been carried out before and is a significant improvement over current methods which involve analysis of mixed and/or isolated bronchial breath. The alternative method for diagnosing oesophagogastric cancer is through biopsy, which is clearly far more invasive and distressing to the patient. Despite the potential advantage of VOC sampling directly from the lumen adjacent to the lesion, until now, no method has been described for the sampling of gas from within the intestinal tract. This is principally because of the technical and logistical challenges of intraluminal gas sampling and subsequent analysis. Recent developments in the technology of mass spectrometry and quality assurance methods of gas analysis have facilitated the development of intraluminal gas analysis.

Earlier detection of oesophagogastric cancer significantly improves survival rates. It will be appreciated that “diagnosis” can mean the initial identification of the nature of an illness or condition, and that “prognosis” can mean predicting the rate of progression or improvement and/or duration of the condition. A prognostic method may be performed subsequent to, and separately from, an initial diagnosis.

Preferably, the endoluminal sample is a gas sample. Preferably, the endoluminal sample comprises an oesophago-gastric endoluminal sample, more preferably an oesophago-gastric endoluminal head space sample. Preferably, the endoluminal sample is obtained from within the lumen of the stomach or oesophagus. Preferably, the endoluminal sample is obtained at least adjacent to a tumour, or suspected location of a tumour, in the test subject. Preferably, the endoluminal sample is obtained within 200 mm or 170 mm of a tumour, or suspected location of a tumour, in the test subject. Preferably, the endoluminal sample is obtained within 150 mm or 120 mm of a tumour, or suspected location of a tumour, in the test subject. Preferably, the endoluminal sample is obtained within 100 mm or 75 mm of a tumour, or suspected location of a tumour, in the test subject. Preferably, the endoluminal sample is obtained within 50 mm or 40 mm of a tumour, or suspected location of a tumour, in the test subject. Preferably, the endoluminal sample is obtained within 20 mm of a tumour, or suspected location of a tumour, in the test subject. Preferably, the endoluminal sample is obtained within 10 mm of a tumour, or suspected location of a tumour, in the test subject. Preferably, the endoluminal sample is obtained within 5 mm, 4 mm, 3 mm, 2 mm or 1 mm of a tumour, or suspected location of a tumour, in the test subject.

The volume of the endoluminal sample may be at least about 50 ml, 100 ml, or 200 ml. Preferably, the volume of the endoluminal sample is at least about 300 ml, 400 ml, or 500 ml. Preferably, the volume of the endoluminal sample is less than about 1000 ml, 850 ml, or 600 ml. Preferably, the volume of the endoluminal sample is between about 50 ml and 1000 ml, or between 100 ml and 850 ml, or between 200 ml and 600 ml.

The apparatus of the third or sixth aspect may comprise sample extraction means for obtaining the endoluminal sample from the test subject. The sample extraction means may comprise a sampling tube, or the like, which is configured to obtain the endoluminal sample from the test subject. The sampling tube may be between 1 mm and 5 mm in diameter. A distal end of the sampling tube is configured to receive the endoluminal sample. A proximal end of the sampling tube is preferably connected to a thermal desorption (TD) tube.

The apparatus may comprise a sample collection container for receiving the extracted sample. The apparatus may further comprise instructions for use.

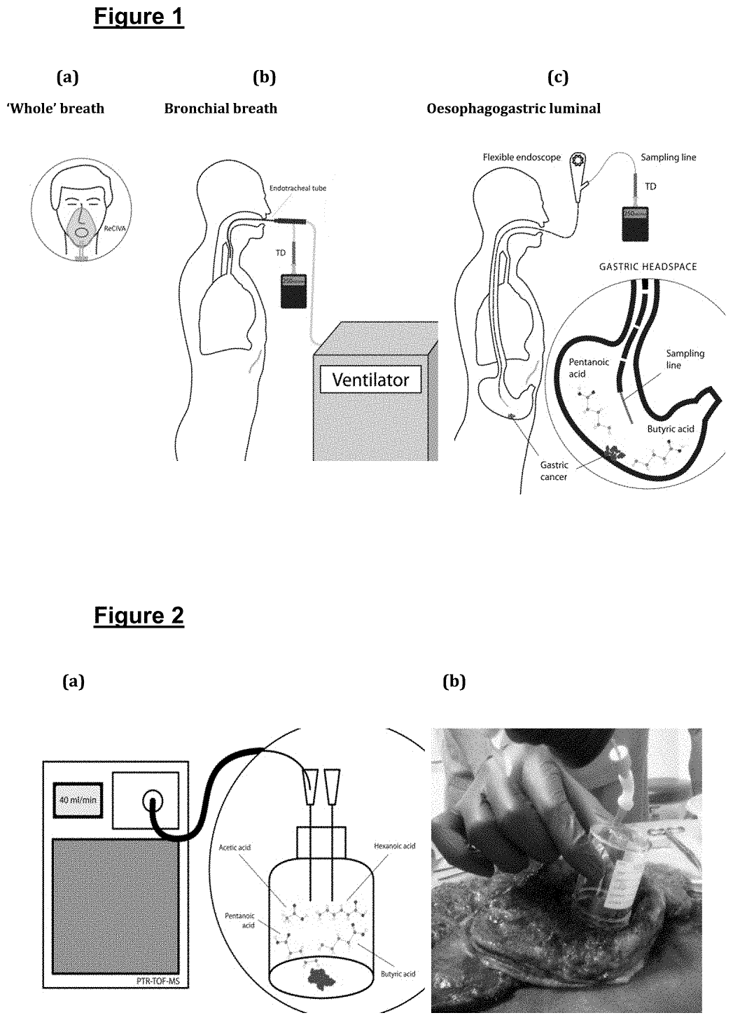

As shown in FIG. 1(c), in one embodiment, the inventors have developed a novel apparatus and method to obtain an endoluminal sample through the operating channel of a flexible endoscope. Accordingly, the apparatus may comprise an endoscope and catheter attached thereto, wherein the catheter is configured to obtain the endoluminal sample from the test subject. The apparatus may comprise a sample catheter and a fluid trap. The apparatus may comprise means for connecting the TD tube in series with an aspiration pump. In another embodiment, the apparatus may comprise a nasogastric tube, which is configured to obtain the endoluminal sample from the test subject.

In some embodiments, the nasogastric tube or the endoscope (preferably the catheter) may comprise one or more sensor configured to determine the level of at least one biomarker compound consisting of: acetone, acetic acid, butyric acid, pentanoic acid and hexanoic acid, in the endoluminal sample. Advantageously, such sensors can provide immediate results. Examples of suitable detector for detecting the at least one biomarker compound preferably include an electrochemical sensor, a semiconducting metal oxide sensor, a quartz crystal microbalance sensor, an optical dye sensor, a fluorescence sensor, a conducting polymer sensor, a composite polymer sensor, or optical spectrometry.

The method may comprise inflating the stomach with medical air (e.g. containing 21% oxygen and 79% nitrogen). The method may then comprise advancing the sampling tube for obtaining the endoluminal sample into the lumen, preferably during endoscopy.

Preferably, the apparatus comprises a pump which is configured to withdraw air from the upper gastrointestinal tract, preferably into thermal desorption tubes, for subsequent biomarker compound analysis. Preferably, the pump is configured to suck the endoluminal sample from the endoluminal space of the test subject. The suction rate of the pump may be at least about 25 ml/min, 50 ml/min, 100 ml/min, or 200 ml/min. Preferably, the suction rate of the pump may be less than about 1000 ml/min, 850 ml/min, or 600 ml/min, or 400 ml/min, or 300 ml/min. Preferably, the suction rate of the pump may be between about 50 ml/min and 500 ml/min, or between 100 ml/min and 400 ml/min, or between 200 ml/min and 300 ml/min.

At least one biomarker compound is preferably a volatile organic compound (VOC), which leads to a fermentation profile, and may be detected in the bodily sample by a variety of techniques. In a preferred embodiment, at least one biomarker compound is detected from gas or vapour. For example, as the signature compounds are VOCs, they may emanate from, or form part of, the endoluminal sample, and may thus be detected in gaseous or vapour form. Thus, these compounds may be detected using a gas analyser. Examples of suitable detector for detecting the at least one biomarker compound preferably include an electrochemical sensor, a semiconducting metal oxide sensor, a quartz crystal microbalance sensor, an optical dye sensor, a fluorescence sensor, a conducting polymer sensor, a composite polymer sensor, or optical spectrometry.

The inventors have demonstrated that the biomarker compounds can be reliably detected using gas chromatography, mass spectrometry, GCMS and/or TOF. Dedicated sensors could be used for the detection step, however.

In one preferred embodiment, the endoluminal sample is analysed using gas chromatography. Furthermore, the level of the at least one biomarker compound is preferably determined by mass spectrometry. Most preferably, GC-MS is used. In another embodiment, the endoluminal sample is analysed TR-Tof-MS.

Preferably, the detection or diagnostic/prognostic method is performed in vitro. Preferably, the sample analysis is performed in vitro. It will also be appreciated that “fresh” bodily samples may be analysed immediately after they have been taken from a subject. Alternatively, the samples may be stored and analysed at a later date. However, in other embodiments, the analysis may be performed in vivo, i.e. real-time.

MS data of the separated VOC components may be compared with NIST Mass Spectral Library version 2.0 for identification of the biomarker compounds including: acetic acid, propanoic acid, butyric acid, pentanoic acid and hexanoic acid. Analysis may be performed for the biomarker compounds presented in Table 1.

It will be appreciated that any one of the biomarkers selected from the group consisting of acetone, acetic acid, butyric acid, pentanoic acid and hexanoic acid may be detected. However, preferably the level of at least two biomarker compounds selected from acetone, acetic acid, butyric acid, pentanoic acid and hexanoic acid may be determined. More preferably, the level of at least three biomarker compounds selected from acetone, acetic acid, butyric acid, pentanoic acid and hexanoic acid may be determined. Even more preferably, the level of at least four or five biomarker compounds selected from acetone, acetic acid, butyric acid, pentanoic acid and hexanoic acid may be determined. In some embodiments, acetone levels may not be determined. Hence, the level of at least one, two, three or four biomarker compounds selected from acetic acid, butyric acid, pentanoic acid and hexanoic acid may be determined.

In an embodiment, the oesophagogastric cancer is selected from gastric cancer, oesophageal cancer, oesophageal squamous-cell carcinoma (ESCC), and oesophageal adenocarcinoma (EAC).

The methods of the invention are useful for monitoring the efficacy of a putative treatment for the oesophagogastric cancer. For example, the treatment for resectable oesophagogastric cancer may comprise neoadjuvant chemotherapy, or chemoradiotherapy followed by surgery and adjuvant chemotherapy. The treatment for very early stage oesophagogastric cancer may comprise endoscopic resection. The treatment for advanced oesophagogastric cancer may comprise palliative chemotherapy.

Preferably, the endoluminal sample is taken from the subject, and at least one biomarker compound in the bodily sample is then detected. In some embodiments, the concentration of at least one biomarker compound is measured.

It will be appreciated that the concentration of at least one biomarker compound in patients suffering from the oesophagogastric cancer is highly dependent on a number of factors, for example how far the disease has progressed, and the age and gender of the subject. It will also be appreciated that the reference concentration of the biomarker compound in individuals who do not suffer from oesophagogastric cancer may fluctuate to some degree, but that on average over a given period of time, the concentration tends to be substantially constant. In addition, it should be appreciated that the concentration of at least one biomarker compound in one group of individuals who suffer from oesophagogastric cancer may be different to the concentration of that compound in another group of individuals who do not suffer from oesophagogastric cancer. However, it is possible to determine the average concentration of at least one biomarker compound in individuals who do not suffer from the oesophagogastric cancer, and this is referred to as the reference or ‘normal’ concentration of biomarker compound. The normal concentration corresponds to the reference values discussed above.

In one embodiment, the methods of the invention preferably comprise determining the ratio of chemicals within the endoluminal sample (i.e. use other components within it as a reference), and then compare these markers to the disease to show if they are elevated or reduced. The reference values may be obtained by assaying a statistically significant number of control samples (i.e. samples from subjects who do not suffer from oesophagogastric cancer). Accordingly, the reference (ii) described herein may be a control sample (for assaying).

The apparatus preferably comprises a positive control (most preferably provided in a container), which corresponds to the at least one biomarker compound(s). The apparatus preferably comprises a negative control (preferably provided in a container). In a preferred embodiment, the apparatus may comprise the reference, a positive control and a negative control. The apparatus may also comprise further controls, as necessary, such as “spike-in” controls to provide a reference for concentration, and further positive controls for each of the biomarker compounds.

Accordingly, the inventors have realised that the difference in concentrations of at least one biomarker compound between the reference normal (i.e. control) and increased/decreased levels, can be used as a physiological marker, suggestive of the presence of oesophagogastric cancer in the test subject. It will be appreciated that if a subject has an increased/decrease concentration of one or more signature compounds which is considerably higher/lower than the ‘normal’ concentration of that compound in the reference, control value, then they would be at a higher risk of having the disease, or a condition that was more advanced (i.e. a negative prognosis), than if the concentration of that compound was only marginally higher/lower than the ‘normal’ concentration.

A skilled technician will appreciate how to measure the concentrations of the biomarker compound in a statistically significant number of control individuals, and the concentration of the same biomarker compound in the test subject, and then use these respective figures to determine whether the test subject has a statistically significant increase/decrease in the compound's concentration, and therefore infer whether that subject is suffering from the disease for which they are being screened.

The subject may be any animal of veterinary interest, for instance, a cat, dog, horse etc. However, it is preferred that the subject is a mammal, such as a human, either male or female.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1(a) shows pre-procedure ‘whole’ breath samples collection from a patient with a ReCIVA device, (b) shows intra-operative sampling of the bronchial breath via the endotracheal tube. (TD, thermal desorption tube), and (c) shows sampling of the oesophagogastric luminal headspace via a suction channel of a standard endoscope with a custom made catheter directly adjacent to the tumour. (TD, thermal desorption tube). FIG. 1(c) shows the biomarkers butyric acid and pentanoic acid in the subject's gut;

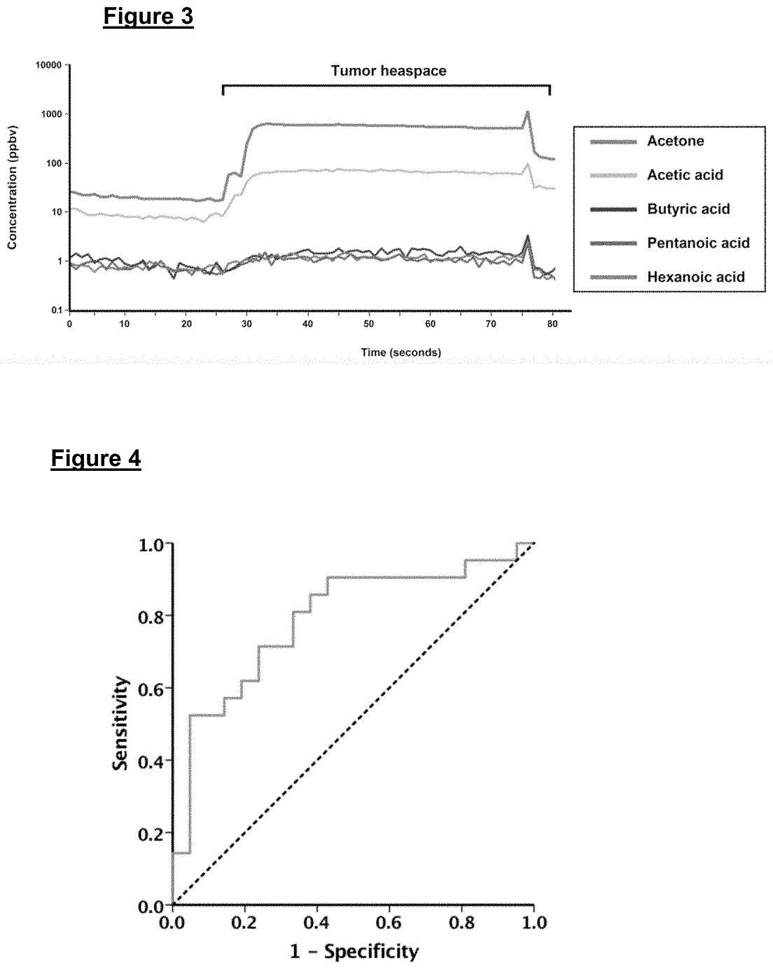

FIG. 2 (a) shows ex vivo headspace analysis with PTR-ToF-MS, and (b) shows direct PTR-ToF-MS analysis of the headspace of cancer and healthy tissue regions of a surgically resected stomach;

FIG. 3 shows a PTR-ToF mass spectrum of direct sampling from a fresh gastric cancer specimen;

FIG. 4 shows a Receiver Operating Characteristic curve (ROC) for volatile fatty acids significant on univariate analysis (for butyric acid and pentatonic acid); and

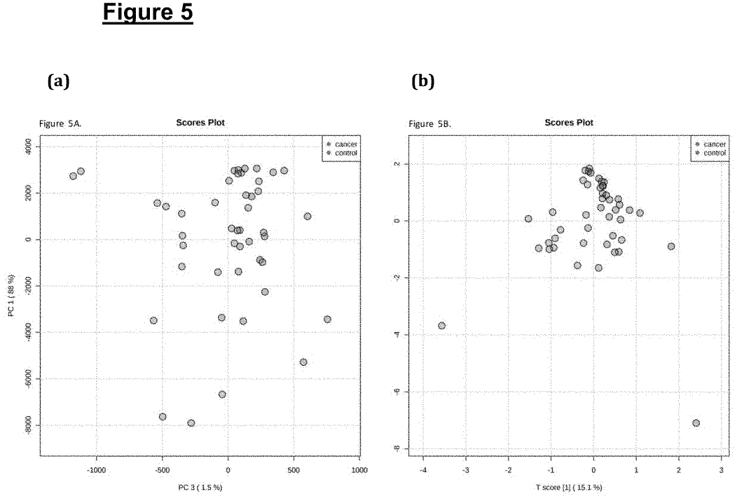

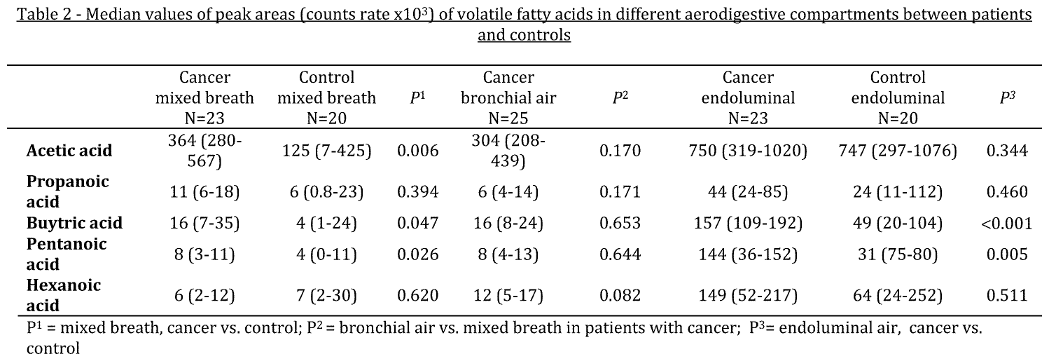

FIG. 5 shows Principal Component Analysis (a) and Orthogonal Partial Least Square (b) of endoluminal volatile fatty acids in cancer patients and control.

### EXAMPLES

The inventors investigated the use of gas biopsy of volatile organic compounds (VOCs) from the oesophago-gastric endoluminal space for the detection of oesophagogastric cancer.

### Materials & Methods

### Study Population

Subjects were recruited from St Mary's Hospital, Imperial College Healthcare NHS Trust in 2017. Comparative analysis of tissue headspace VOCs was performed in patients with: (i) biopsy proven oesophagogastric cancer; non-cancer disease of the upper gastrointestinal tract (e.g., esophagitis, gastritis and peptic ulcer disease), and; healthy upper gastrointestinal tract (normal appearance on endoscopy) with a negative rapid urease test for the Helicobacter pylori. All patients were required to be fasted and to refrain from smoking for a minimum of six hours prior to breath testing. Patients were excluded if they had known liver disease, small bowel/colonic conditions or a synchronous cancer at another site. Local ethics committee approval through NHS Health Research Authority was granted for this study (Ref: 15/LO/1140) and written informed consent was obtained from all patients prior to enrolment in the study.

Targeted Analysis of Volatile Fatty Acids within Separate In Vivo Compartments

The inventors performed targeted in vivo analysis of VOCs within three anatomical compartments: (i) ‘whole’ breath; (ii) isolated bronchial breath, and (iii) the oesophagogastric luminal headspace.

### Analysis of ‘Whole Breath’

Referring to FIG. 1(a), prior to either upper gastrointestinal endoscopy and/or elective surgery, a 500 mL ‘whole’ breath sample was collected using a ReCIVA breath sampler (Owlstone, Cambridge, UK) in an accordance with an established methodology (FIG. 1a).¹² Briefly, patients were asked to breath tidally into the device through a single use facemask. Exhaled breath was pumped on to four thermal desorption (TD) tubes (Markes International, Ilantrisant, UK) pre-packed with 200 mg of Tenax and 100 mg of Carbograph 5 to a total volume of 500 ml per tube.

### Analysis of In-Vivo Endoluminal Bronchial Gas

Referring to FIG. 1(b), in cancer patients undergoing surgery (staging laparoscopy and upper gastrointestinal endoscopy), a sample of isolated bronchial air was obtained shortly after induction of general anaesthesia and endotracheal intubation. Bronchial air (500 ml) was sampled directly onto TD (thermal desorption) tubes using a handheld precision 210-1002MTX pump (SKC Ltd, Dorset, UK). Breath was sampled from the capnography port of the ventilator circuit throughout the respiratory cycle (FIG. 1b). The following standardised ventilatory settings were applied 5 mins prior to and for the duration of sampling: fraction of inspired oxygen 100%; respiratory rate 10 breaths per minute, and; 5 mmHg positive end expiratory pressure. All traces of volatile anaesthetic gases were removed from the anaesthetic circuit prior to bronchial sampling to avoid their potential influence on breath gas analysis. Total intravenous anaesthesia was induced and maintained using with alfentanil and propofol.

### Analysis of In-Vivo Endoluminal Gastric Gas

Referring to FIG. 1(c), the inventors developed a method to sample gastrointestinal luminal air through the operating channel of a flexible endoscope. After inflation of the stomach with medical air, a 2 mm wide sample line (V-green, Vygon, Paris, France) was advanced in to the gastric lumen during upper gastrointestinal endoscopy. The proximal end of the sampling line was connected to a TD tube and a sample of 500 ml luminal air was obtained at a rate of 250 ml/min using a handheld precision 210-1002MTX pump (FIG. 1c).

### Analysis of In-Vivo Sampling by Thermal Desorption GC-MS

Samples were analysed using an Agilent 7890B GC with 5977A MSD (Agilent Technologies, Cheshire, UK), coupled to a Markes TD-100 device (Markes International, Liantrisant, UK). Prior to sample collection TD tubes were conditioned at 325° C. for 40 minutes in a stream of nitrogen passed through a hydrocarbon trap (Supelco, US) using a Markes International TC-20 tube conditioner (Markes International, Liantrisant, UK). Details of the conditions of analysis using TD-GC-MS have been published elsewhere.¹³ Briefly, TD tube samples were pre-purged for 1 min at 50 mL/min constant helium flow rate prior to 280° C. for 10 min. Following secondary desorption by heating the cold trap (U-T12ME-2S) from 10° C. to 290° C. at 99° C./min and held for 4 min. The GC flow path was heated constantly at 140° C. VOC separation was performed on a ZB-624 capillary column (60 m×0.25 mm ID×1.40 μm df; Phenomenex Inc., Torrance, USA) programmed at 1.0 mL/min constant Helium carrier flow. Oven temperature profile was set at 40° C. initially for 4 min, ramp to 100° C. (5° C./min with 1 min hold), ramp to 110° C. (5° C./min with 1 min hold), ramp to 200° C. (5° C./min with 1 min hold), finally ramp to 240° C. at 10° C./min with 4 min hold. The MS transfer line was maintained at 240° C. whilst 70 eV electron impact at 230° C. was set while the quadruple was held at 150° C. MS analyser was set to acquire over the range of 20 to 250 m/z with data acquisition approximated to 6 scan/sec. GC-MS data was then processed using MassHunter software version B.07 SP1 (Agilent Technologies, Cheshire, UK) while MS data of the separated VOC component was compared with NIST Mass Spectral Library version 2.0 for identification of target compounds including: acetic acid, propanoic acid, butyric acid, pentanoic acid and hexanoic acid.¹⁴

In a single patient, direct headspace analysis of a gastric tumour and adjacent ‘normal’ mucosa was performed immediately after resection of the whole stomach. The purpose of this experiment was to determine VOC levels within localised regions of the stomach (diseased and ‘healthy’) and to perform cross platform validation of results. A sterile polystyrene sample container (60 mL) was modified to permit the passage of the PTR-TOF-MS sample line through its base and was placed over the tumour and the headspace was analysed for 60 seconds. Headspace above adjacent gastric mucosa that was macroscopically uninvolved by tumour was subsequently.

### Analysis by PTR-Tof-MS

A PTR-ToF 1000 mass spectrometer equipped with a commercial SRI feature (Ionicon Analytik GmbH, Innsbruck, Austria) was coupled with a TD autosampler (TD100-xr, Markes International Ltd., Llantrisant, UK) for the analysis. Detailed system setup was described in the inventors' previous work.¹⁵ During the current experiments, a series of quality checks were conducted on the PTR-ToF-MS daily. Quantitative accuracy was within ±10% of a certified standard, represented by a Trace Source™ benzene permeation tube (Kin-Tek Analytical Inc., La Marque TX). When H₃O⁺ was used as the primary ion, O₂⁺ impurities were <2%. Repeatability of fragmentation patterns with H₃O⁺ as primary ions was assessed by measuring the ratio between peaks m/z89 and 71 were used to represent the quasi-molecular and the most representative fragment for butyric acid, as obtained from a permeation tube standard. The values measured on the different days were within ±2% of the mean. When required, the voltage of the microchannel plate and the mass resolution (>1,500 m/δm) was optimised using m/z 89 (butyric acid with H₃O⁺) as reference peak. Data were first extracted using PTRMS viewer version 3.2.2.2 (Ionicon Analytik) and subjected to further analysis using in-house generated scripts written using R-programming language. Target analysis was performed for compounds presented in Table 1.

TABLE 1
A summary of analytical information of compounds detected and
quantified by PTR-ToF-MS using the H₃O⁺ precursor ion

| | Molecular | Precursor | | Characteristic |
|---|---|---|---|---|
| | formula | ions | m/z | product ions |
| Acetone | C₃H₆O | H₃O⁺ | 59.049 | C₃H₆OH⁺ |
| Acetic acid | C₂H₄O₂ | H₃O⁺ | 61.028 | C₂H₄O₂H⁺ |
| Butyric acid | C₄H₈O₂ | H₃O⁺ | 89.060 | C₄H₈O₂H⁺ |
| Pentanoic acid | C₅H₁₀O₂ | H₃O⁺ | 103.075 | C₅H₁₀O₂H⁺ |
| Hexanoic acid | C₆H₁₂O₂ | H₃O⁺ | 117.091 | C₆H₁₂O₂H⁺ |

### Statistical Analysis

Statistical analysis was performed using IBM SPSS statistics 22 (SPSS Inc., Chicago, Ill.) and Prism (Ver. 7.0d, GraphPad Software, San Diego, Calif.). VOCs data (not normally distributed) is presented as a median and interquartile range. The Mann-Whitney U test was use for pairwise comparisons. Principal Component Analysis was performed according to method described by David et al.¹⁶ Receiver operating characteristic (ROC) analysis was performed for VFAs significant on univariate analysis after determining their test probabilities using binominal logistic regression. Unsupervised Principal Component Analysis (PCA) and supervised orthogonal partial least square analysis (OPIS) was performed with MetaboAnalyst 4.0 software (McGill University, Canada). A p-value≤0.05 was taken as the level of statistical significance.

### Methods for Endoluminal Gastric Gas Biopsy

1. As an adjunct method during endoscopy: The same method of collection and analysis as described above to collect air from the gastric lumen. Analysis is carried out using Mass spectrometry. National studies showed that about 9% of gastric and oesophageal cancers were missed during endoscopy prior to diagnosis (30-31).
2. Luminal gas is collected by inserting a fine nasogastric tube and using a pump to withdraw air from the upper gastrointestinal tract into thermal desorption tubes for subsequent lab analysis. Analysis is carried out using Mass spectrometry.
3. Sensors that detect fatty acids biomarkers are mounted on nasogastric tubes or endoscope to provide immediate results.

### Results

Targeted Analysis of Volatile Fatty Acids within Isolated In Vivo Compartments

The ability to interpret VOCs measurements from complex and dynamic biological matrices remains challenging. Technological advances in gas phase analytical techniques permit measurement of VOCs emitted from the headspace of biofluids and histological specimens with accuracy at levels of parts-per-trillion by volume (pptv). In particular, mass spectrometry techniques including Proton-Transfer-Reaction Time-of-Flight Mass Spectrometry (PTR-ToF-MS) and Gas Chromatography Mass Spectrometry (GC-MS) have been widely utilised for VOC detection in human studies.^{10,11} PTR-ToF-MS is notable for its ability to perform real-time analysis of a full mass spectrum within a fraction of a second and with separation and identification of isobaric ions.

In total, 25 patients with oesophagogastric cancer (17 male, 74±14 yrs) and 20 control subjects (10 male, 57±17 Yrs) were recruited. Baseline sampling of mixed breath using the ReCIVA device was completed in all patients and an additional isolated bronchial breath sample was collected in all cancer patients. In two patients, intraluminal gastric headspace sampling was abandoned due to contamination of the sampling line with gastric secretions. The median peak areas of the different VOCs in these compartments are presented in Table 2. ROC analysis for butyric and pentatonic acid gave an area under the curve of 0.80 (95% CI 0.65-0.93; P=0.01) (FIG. 1). Unsupervised PCA and supervised OPIS analysis demonstrated that the examined VFAs contribute to the clustering and discrimination of endoluminal air between cancer and control subjects (FIG. 2).

Compared to mixed and bronchial breath samples, all examined VOCs were found at highest concentrations within the oesophagogastric luminal headspace. In addition, VOCs tended to be higher in all samples derived from cancer patients compared to controls. Butyric acid and pentanoic acid were found to be significantly elevated in the ‘whole’ breath and endoluminal air of cancer patients compared to controls, with endoluminal levels being approximately ten times greater than found in ‘whole’ breath, which was unexpected. Equivalence of volatile fatty acids (VFA) levels within the mixed and isolated bronchial breath of cancer patients suggests that their origin within breath is principally derived from the lungs and by inference the systemic circulation as opposed to direct passage from the upper gastrointestinal tract, as previously proposed. It is noteworthy that whilst acetic acid levels were significantly elevated in the ‘whole’ breath of cancer patients, equivalent enriched levels were found in the endoluminal air of both cancer and control subjects. This could suggest that the raised levels of acetic acid found within the exhaled breath patients with oesophagogastric cancer may be influenced by other, as yet undetermined, systemic sources.

Direct sampling of the headspace of a gastric cancer immediately following surgical resection of the whole stomach was performed in a single patient using PTR-ToF-MS (FIG. 3). Acetone (795.3 vs. 388.8 ppbu), acetic acid (29.0 vs. 18.1 ppbu), butyric acid (2.8 vs 1.8 ppbu), pentanoic acid (1.1 vs 0.8 ppbu) and hexanoic acid (1.7 vs 1.0 ppbu) were observed at higher concentrations within the in-situ headspace above the tumour compared to macroscopically normal adjacent gastric mucosa.

Referring to FIG. 4, there is shown a Receiver Operating Characteristic curve (ROC) for volatile fatty acids significant on univariate analysis (for butyric acid and pentatonic acid).

Referring to FIG. 5, there is shown Principal Component Analysis (a) and Orthogonal Partial Least Square (b) of endoluminal volatile fatty acids in cancer patients and control.

### Discussion

Taken together, the findings support a clear association between cancer and dysregulation of VOC metabolism.^{9,20} Fatty acids are absorbed within the small and large bowel and play an important role in many cellular functions.¹⁷ Fatty acids may contribute to carcinogenesis through cell membrane production, energy metabolism, cell signalling and prevention of apoptosis.²¹ In human malignancies, including gastric cancer, overexpression of fatty acid synthase leads to increased de novo synthesis of fatty acids and is associated with poor prognosis.¹⁸⁻²⁰

Acetic acid is a metabolic intermediate within the pathway of acetyl-CoA synthesis. In the inventors' previous studies of gastric content and urine, they observed higher concentrations of acetic acid in oesophagogastric cancer patients compared to healthy controls.^{4,5} Zhang et al. performed NMR spectroscopy of blood samples from patients with oesophageal adenocarcinoma and reported that changes in the trichloroacetic acid cycle were dominant factors in the biochemistry of this cancer.²¹ Hasim et al. also reported increased levels of acetate in the NMR profile of urine in patients with oesophageal cancer compared to healthy controls.²²

In a recent multicenter validation study investigating exhaled breath analysis for oesophagogastric cancer, butyric acid was identified as a key discriminatory VOC.²³ Shi et al. also reported that 4-phenybutyric acid promotes gastric cancer cell migration via histone deacetylase mediated HER3/HER4 upregulation.²⁴ Butyric acid can also be produced from periodontopathic bacteria as an extracellular metabolite and it has been implicated in the development of oral cancer.¹⁷

Pentanoic acid is an aliphatic fatty acid that has an important role in tumorgenesis.²⁵ Moreover, both pentanoic acid and hexanoic acid were principal VOCs in the exhaled breath diagnostic prediction models for oesophagogastric cancer in both the initial studies and a subsequent multicenter study.^{4,23} Using TD-GCxGC-ToF-MS, Stadler et al. identified hexanoic acid as a potential marker of tissue necrosis and decomposition in cadavers.²⁶ Accordingly, hexanoic acid may be released in higher amounts within regions of necrosis in oesophagogastric tumours. Hexanoic acid has also been reported to be significantly increased in the plasma of patients with high-grade dysplastic colonic adenomas compared to controls.

Acetone and other ketone bodies are thought to permit sustaining abnormal tumour growth by acting as an alternative energy sources.^{27,28} Acetone is produced through lipolysis or from acetyl-CoA as a breakdown product of fatty acid oxidation. The inventors previously observed higher concentrations of acetone within the gastric content and urine of oesophagogastric cancer patients compared to controls.^{4,5} Hasim et al. have reported significantly increased blood plasma acetone concentrations in patients with poorly differentiated oesophageal cancer.²² Ketones may function as chemo-attractants and stimulate the migration of epithelial cancer cells stimulating primary tumour growth.²⁹

In the face of growing evidence for the association between VOCs and deregulated tumour metabolism, the mechanism whereby they are released in to exhaled breath remains relevant, but incompletely understood. There are thought to be two main pathways by which VOCs may partition between the body and exhaled breath, i.e. through passage from the systemic circulation across the alveolar capillary barrier or via direct release from the upper airways and digestive tract.⁹ Importantly, this study has been able to measure isolated bronchial breath in intubated cancer patients. Whilst acknowledging inconsistencies in the methods used to assess breath from patients who were intubated or breathing spontaneously, the observed general consistency in the levels of exhaled VOCs within these compartments has two principal implications. Firstly, whilst these VOCs may be concurrently found in relative abundance within the upper gastrointestinal endoluminal air, unexpectedly this does not appear to be a source of significant contamination of exhaled breath. Secondly, if the tumour is indeed the source of these VOCs in exhaled breath, the process whereby they are transported to the lung within the systemic circulation before being partitioned across the alveolar capillary barrier leads to a significant attenuation in their detectable levels.

### Clinical Implications

There are diagnostic clinical implications of these studies. The marked difference in VOCs levels in endoluminal gastro-oesophageal air of cancer compared to control patients provides the surprising opportunity of using endoluminal gas biopsy for cancer detection instead of detection of the VOCs in exhaled breath. Secondly, the non-significant difference between exhaled and isolated bronchial breath supports the use of mixed exhaled breath for non-invasive cancer detection without the need for complex devices for alveolar sampling.

### CONCLUSIONS

The invention described herein is a method and associated apparatus for measuring airborne VOCs to diagnose a disease state, such as oesophagogastric cancer. Although readings can be taken from the breath or bronchial air, the best results are clearly obtained from the oesophagogastric luminal headspace. The samples are analysed by mass spectrometry to find the concentration of volatile fatty acids that are known to be biomarkers for cancer. The inventors are the first to use this diagnostic method inside the oesophagogastric lumen to attain a more concentrated air sample for the diagnosis of oesophagogastric cancer.

### REFERENCES

(1) Amal, H.; Leja, M.; Funka, K.; Skapars, R.; Sivins, A.; Ancans, G.; Liepniece-Karele, I.; Kikuste, I.; Lasina, I.; Haick, H. Gut 2016, 65, 400-407.
(2) Kumar, S.; Huang, J.; Abbassi-Ghadi, N.; Mackenzie, H. A.; Veselkov, K. A.; Hoare, J. M.; Lovat, L. B.; Spanel, P.; Smith, D.; Hanna, G. B. Ann Surg 2015, 262, 981-990.
(3) Kumar, S.; Huang, J.; Abbassi-Ghadi, N.; Spanel, P.; Smith, D.; Hanna, G. B. Anal Chem 2013, 85, 6121-6128.
(4) Kumar, S.; Huang, J.; Cushnir, J. R.; Spanel, P.; Smith, D.; Hanna, G. B. Anal Chem 2012, 84, 9550-9557.
(5) Huang, J.; Kumar, S.; Abbassi-Ghadi, N.; Spanel, P.; Smith, D.; Hanna, G. B. Anal Chem 2013, 85, 3409-3416.
(6) Litvak, D. A.; Hwang, K. O.; Evers, B. M.; Townsend, C. M., Jr. Anticancer Res 2000, 20, 779-784.
(7) Okabe, S.; Okamoto, T.; Zhao, C. M.; Chen, D.; Matsui, H. J Gastroenterol Hepatol 2014, 29 Suppl 4, 65-69.
(8) Tait, E.; Perry, J. D.; Stanforth, S. P.; Dean, J. R. J Chromatogr Sci 2014, 52, 363-373.
(9) Haick, H.; Broza, Y. Y.; Mochalski, P.; Ruzsanyi, V.; Amann, A. Chem Soc Rev 2014, 43, 1423-1449.
(10) Zhan, X.; Duan, J.; Duan, Y. Mass Spectrom Rev 2013, 32, 143-165.
(11) Smith, D.; Spanel, P. Mass Spectrom Rev 2005, 24, 661-700.
(12) Doran, S.; Romano, A.; Hanna, G. B. J Breath Res 2017.
(13) Chin, S. T.; Romano, A.; Doran, S. L. F.; Hanna, G. B. Sci Rep 2018, 8, 5139.
(14) Wei, X.; Koo, I.; Kim, S.; Zhang, X. Analyst 2014, 139, 2507-2514.
(15) Romano, A.; Doran, S.; Belluomo, I.; Hanna, G. B. Anal Chem 2018, 90, 10204-10210.
(16) David, C. C.; Jacobs, D. J. Methods Mol Biol 2014, 1084, 193-226.
(17) Layden, B. T.; Angueira, A. R.; Brodsky, M.; Durai, V.; Lowe, W. L., Jr. Tansl Res 2013, 161, 131-140.
(18) Migita, T.; Ruiz, S.; Fornari, A.; Fiorentino, M.; Priolo, C.; Zadra, G.; Inazuka, F.; Grisanzio, C.; Palescandolo, E.; Shin, B.; Fiore, C.; Xie, W.; Kung, A. L.; Febbo, P. G.; Subramanian, A.; Mucci, L.; Ma, J.; Signoretti, S.; Stampfer, M.; Hahn, W. C., et al. J Natl Cancer Inst 2009, 101, 51-532.
(19) Carvalho, M. A.; Zecchin, K. G.; Seguin, F.; Bastos, D. C.; Agostini, M.; Rangel, A. L.; Veiga, S. S.; Raposo, H. F.; Oliveira, H. C.; Loda, M.; Coletta, R. D.; Graner, B. Int J Cancer 2008, 123, 2557-2565.
(20) Rossi, S.; Graner, B.; Febbo, P.; Weinstein, L.; Bhattacharya, N.; Onody, T.; Bubley, G.; Balk, S.; Loda, M. Mol Cancer Res 2003, 1, 707-715.
(21) Zhang, J.; Liu, L.; Wei, S.; Nagana Gowda, G. A.; Hammoud, Z.; Kesler, K. A.; Raftery, D. J Thorac Cardiovasc Surg 2011, 141, 469-475, 475 e461-464.
(22) Hasim, A.; Ma, H.; Mamtimin, B.; Abudula, A.; Niyaz, M.; Zhang, L. W.; Anwer, J.; Sheyhidin, I. Mol Biol Rep 2012, 39, 8955-8964.
(23) Markar, S. R.; Wiggins, T.; Antonowicz, S.; Chin, S. T.; Romano, A.; Nikolic, K.; Evans, B.; Cunningham, D.; Mughal, M.; Lagergren, J.; Hanna, G. B. JAMA Oncol 2018.
(24) Shi, X.; Zheng, C.; Li, C.; Hou, K.; Wang, X.; Yang, Z.; Liu, C.; Liu, Y.; Che, X.; Qu, X. Cell Biol Int 2018, 42, 53-62.
(25) Haddad, J. J. Recent Pat Inflamm Allergy Drug DiscoV 2013, 7, 229-258.
(26) Crotti, S.; Agnoletto, B.; Cancemi, G.; Di Marco, V.; Traldi, P.; Pucciarelli, S.; Nitti, D.; Agostini, M. Anal Bioanal Chem 2016, 408, 6321-6328.
(27) Puchalska, P.; Crawford, P. A. Cell Metab 2017, 25, 262-284.
(28) Schug, Z. T.; Peck, B.; Jones, D. T.; Zhang, Q.; Grosskurth, S.; Alam, I. S.; Goodwin, L. M.; Smethurst, E.; Mason, S.; Blyth, K.; McGarry, L.; James, D.; Shanks, B.; Kalna, G.; Saunders, R. B.; Jiang, M.; Howell, M.; Lassailly, F.; Thin, M. Z.; Spencer-Dene, B., et al. Cancer Cell 2015, 27, 57-71.
(29) Bonuccelli, G.; Tsirigos, A.; Whitaker-Menezes, D.; Pavlides, S.; Pestell, R. G.; Chiavarina, B.; Frank, P. G.; Flomenberg, N.; Howell, A.; Martinez-Outschoorn, U. E.; Sotgia, F.; Lisanti, M. P. Cell Cycle 2010, 9, 3506-3514.
(30) Chadwick G, Groene O, Riley S, Hardwick R, Crosby T, Hoare J, Hanna G B, Greenaway K, Cromwell D A. Clin Gastroenterol Hepatol. 2015, 13(7):1264-1270.e1.
(31) Chadwick G, Groene O, Hoare J, Hardwick R H, Riley S, Crosby T D, Hanna G B, Cromwell D A. Endoscopy. 2014, 46(7):553-60.

1. A method for diagnosing a subject suffering from oesophagogastric cancer, or a pre-disposition thereto, or for providing a prognosis of the subject's condition, the method comprising analysing, in an endoluminal sample obtained from a test subject, the level of at least one biomarker compound selected from the group consisting of: acetone, acetic acid, butyric acid, pentanoic acid and hexanoic acid, and comparing this level with a reference for the level of the at least one biomarker compound in an individual who does not suffer from oesophagogastric cancer, wherein an increase in the concentration of the at least one biomarker compound, in the endoluminal sample from the test subject, compared to the reference, suggests that the subject is suffering from oesophagogastric cancer, or has a pre-disposition thereto, or provides a negative prognosis of the subject's condition.

2. A method for determining the efficacy of treating a subject suffering from oesophagogastric cancer with a therapeutic agent or a specialised diet, the method comprising analysing, in an endoluminal sample obtained from a test subject, the level of at least one biomarker compound selected from the group consisting of: acetone, acetic acid, butyric acid, pentanoic acid and hexanoic acid, and comparing this level with a reference for the level of at least one biomarker compound in an individual who does not suffer from oesophagogastric cancer, wherein:
(i) a decrease in the level of the at least one biomarker compound in the endoluminal sample from the test subject, compared to the reference, suggests that the treatment regime with the therapeutic agent or the specialised diet is effective, or
(ii) an increase in the concentration of at least one biomarker compound in the endoluminal sample from the test subject, compared to the reference, suggests that the treatment regime with the therapeutic agent or the specialised diet is ineffective.

3. A method according to any preceding claim, wherein the endoluminal sample is a gas sample.

4. A method according to any preceding claim, wherein the endoluminal sample comprises an oesophago-gastric endoluminal sample.

5. A method according to any preceding claim, wherein the endoluminal sample comprises an oesophago-gastric endoluminal head space sample.

6. A method according to any preceding claim, wherein the endoluminal sample is obtained from within the lumen of the stomach or oesophagus.

7. A method according to any preceding claim, wherein the endoluminal sample is obtained at least adjacent to a tumour, or suspected location of a tumour, in the test subject, optionally within 200 mm, 170 mm, 150 mm, or 120 mm of a tumour, or suspected location of a tumour, in the test subject.

8. A method according to any preceding claim, wherein the endoluminal sample is obtained within 100 mm, 75 mm, 50 mm, 40 mm or 20 mm of a tumour, or suspected location of a tumour, in the test subject.

9. A method according to any preceding claim, wherein the volume of the endoluminal sample is at least about 50 ml, 100 ml, or 200 ml, and/or less than about 1000 ml, 850 ml, or 600 ml, optionally between about 50 ml and 1000 ml, or between 100 ml and 850 ml, or between 200 ml and 600 ml.

10. A method according to any preceding claim, wherein the method comprises inflating the stomach with medical air, and then advancing a sampling tube for obtaining the endoluminal sample into the lumen, preferably during endoscopy.

11. A method according to any preceding claim, wherein the at least one biomarker compound is a volatile organic compound (VOC), which is detected in the endoluminal sample.

12. A method according to any preceding claim, wherein the at least one biomarker compound is detected using a gas analyser, optionally wherein the detector for detecting the at least one biomarker compound is an electrochemical sensor, a semiconducting metal oxide sensor, a quartz crystal microbalance sensor, an optical dye sensor, a fluorescence sensor, a conducting polymer sensor, a composite polymer sensor, or optical spectrometry.

13. A method according to any preceding claim, wherein the at least one biomarker compound is detected using gas chromatography, mass spectrometry, GCMS and/or TOF.

14. A method according to any preceding claim, wherein the endoluminal sample is analysed using GC-MS.

15. A method according to any preceding claim, wherein the endoluminal sample is analysed using TR-Tof-MS.

16. A method according to any preceding claim, wherein the level of at least two, three, four or five biomarker compounds selected from acetone, acetic acid, butyric acid, pentanoic acid and hexanoic acid is determined.

17. A method according to any preceding claim, wherein the level of at least one, two, three or four biomarker compounds selected from acetic acid, butyric acid, pentanoic acid and hexanoic acid is determined.

18. A method according to any preceding claim, wherein the oesophagogastric cancer is selected from gastric cancer, oesophageal cancer, oesophageal squamous-cell carcinoma (ESCC), and oesophageal adenocarcinoma (EAC).

19. A method according to any preceding claim, wherein the method is useful for monitoring the efficacy of a putative treatment for the oesophagogastric cancer, optionally wherein: (i) the treatment for resectable oesophagogastric cancer comprises neoadjuvant chemotherapy, or chemoradiotherapy followed by surgery and adjuvant chemotherapy; (ii) the treatment for very early stage oesophagogastric cancer comprises endoscopic resection; or (iii) the treatment for advanced oesophagogastric cancer comprises palliative chemotherapy.

20. An apparatus for diagnosing a subject suffering from oesophagogastric cancer, or a pre-disposition thereto, or for providing a prognosis of the subject's condition, the apparatus comprising:—
means for analysing, in an endoluminal sample obtained from a test subject, the level of at least one biomarker compound selected from the group consisting of: acetone, acetic acid, butyric acid, pentanoic acid and hexanoic acid; and
a reference for the concentration of the at least one biomarker compound in a sample from an individual who does not suffer from oesophagogastric cancer,
wherein the apparatus is used to identify an increase in the concentration of the at least one biomarker compound in the endoluminal sample from the test subject, compared to the reference, thereby suggesting that the subject suffers from oesophagogastric cancer, or has a pre-disposition thereto, or provides a negative prognosis of the subject's condition.

21. An apparatus for determining the efficacy of treating a subject suffering from oesophagogastric cancer with a therapeutic agent or a specialised diet, the apparatus comprising:—
means for analysing, in an endoluminal sample obtained from a test subject, the level of at least one biomarker compound selected from the group consisting of: acetone, acetic acid, butyric acid, pentanoic acid and hexanoic acid; and
a reference for the concentration of the at least one biomarker compound in a sample from an individual who does not suffer from oesophagogastric cancer,
wherein the apparatus is used to identify:
(i) a decrease in the level of the at least one biomarker compound in the endoluminal sample from the test subject, compared to the reference, thereby suggesting that the treatment regime with the therapeutic agent or the specialised diet is effective; or
(ii) an increase in the concentration of the at least one biomarker compound in the endoluminal sample from the test subject, compared to the reference, thereby suggesting that the treatment regime with the therapeutic agent or the specialised diet is ineffective.

22. An apparatus according to either claim 20 or claim 21, wherein the apparatus comprises sample extraction means for obtaining the endoluminal sample from the test subject.

23. An apparatus according to claim 22, wherein the sample extraction means comprises a sampling tube configured to obtain the endoluminal sample from the test subject, optionally wherein the sampling tube is between 1 mm and 5 mm in diameter.

24. An apparatus according to claim 23, wherein a distal end of the sampling tube is configured to receive the endoluminal sample and/or a proximal end of the sampling tube is connected to a thermal desorption (TD) tube.

25. An apparatus according to any one of claims 20-24, wherein the apparatus comprises an endoscope and catheter attached thereto, wherein the catheter is configured to obtain the endoluminal sample from the test subject.

26. An apparatus according to claim 25, wherein the apparatus comprises a fluid trap, optionally wherein the apparatus comprises means for connecting the TD tube in series with an aspiration pump.

27. An apparatus according to any one of claims 20-24, wherein the apparatus comprises a nasogastric tube, which is configured to obtain the endoluminal sample from the test subject.

28. An apparatus according to any one of claims 25-27, wherein the nasogastric tube or the endoscope, optionally the catheter thereof, comprises one or more sensor configured to determine the level of at least one biomarker compound consisting of: acetone, acetic acid, butyric acid, pentanoic acid and hexanoic acid, in the endoluminal sample.

29. An apparatus according to any one of claims 20-28, wherein the apparatus comprises a detector for detecting the at least one biomarker compound, the detecting being an electrochemical sensor, a semiconducting metal oxide sensor, a quartz crystal microbalance sensor, an optical dye sensor, a fluorescence sensor, a conducting polymer sensor, a composite polymer sensor, or optical spectrometry.

30. An apparatus according to any one of claims 20-29, wherein the apparatus comprises a pump which is configured to withdraw air from the upper gastrointestinal tract, preferably into thermal desorption tubes, for subsequent biomarker compound analysis.

31. An apparatus according to claim 30, wherein the pump is configured to suck the endoluminal sample from the endoluminal space of the test subject and the suction rate of the pump is at least about 25 ml/min, 50 ml/min, 100 ml/min, or 200 ml/min and/or less than about 1000 ml/min, 850 ml/min, or 600 ml/min, or 400 ml/min, or 300 ml/min.

32. An apparatus according to any one of claims 20-31, wherein the apparatus comprises a positive control, which corresponds to the at least one biomarker compound(s) and/or a negative control.

33. Use of at least one biomarker compound selected from the group consisting of: acetone, acetic acid, butyric acid, pentanoic acid and hexanoic acid in or from an endoluminal sample obtained from a test subject, as a biomarker for diagnosing a subject suffering from oesophagogastric cancer, or a pre-disposition thereto, or for providing a prognosis of the subject's condition.
